SEQUENCE LISTING (9 sequences)

SEQ ID NO: 1 - protein, 31 aa, Artificial Sequence
  Description of Artificial Sequence Synthetic polypeptide
FNKEQQNAFYEILHLPNLNEEQRNAFIQSLK

SEQ ID NO: 2 - protein, 323 aa, Artificial Sequence
  Description of Artificial Sequence Synthetic polypeptide
MEFGLSWLFLVAILKGVQCFNKEQQNAFYEILHLPNLNEEQRNAFIQSLKGGGGSGGGGSGGGGSTSITLDLVNPTAGQYSSFVDKIRNNVKDPNLKYGGTDIAVIGPPSKEKFLRINFQSSRGTVSLGLKRDNLYVVAYLAMDNTNVNRAYYFRSEITSAESTALFPEATTANQKALEYTEDYQSIEKNAQITQGDQSRKELGLGIDLLSTSMEAVNKKARVVKDEARFLLIAIQMTAEAARFRYIQNLVIKNFPNKFNSENKVIQFEVNWKKISTAIYGDAKNGVFNKDYDFGFGKVRQVKDLQMGLLMYLGKPKHHHHHH

SEQ ID NO: 3 - DNA, 972 bp, Artificial Sequence
  Description of Artificial Sequence Synthetic polynucleotide
atggagtttgggctgagctggctttttcttgtggctattttaaaaggtgtccagtgtttcaacaaagaacaacaaaacgccttctatgaaatcttgcacttgcctaacttgaacgaagaacaacgcaatgctttcatccaaagcttaaaaggtggaggcggttcaggtggaggcggttcaggtggaggcggttcaacatcaatcacattagatctagtaaatccgaccgcgggtcaatactcatcttttgtggataaaatccgaaacaacgtaaaggatccaaacctgaaatacggtggtaccgacatagccgtgataggcccaccttctaaagaaaaattccttagaattaatttccaaagttcccgaggaacggtctcacttggcctaaaacgcgataacttgtatgtggtcgcgtatcttgcaatggataacacgaatgttaatcgggcatattacttcagatcagaaattacttccgccgagtcaaccgcccttttcccagaggccacaactgcaaatcagaaagctttagaatacacagaagattatcagtcgattgaaaagaatgcccagataacacaaggagatcaaagtagaaaagaactcgggttggggattgacttactttcaacgtccatggaagcagtgaacaagaaggcacgtgtggttaaagacgaagctagattccttcttatcgctattcagatgacggctgaggcagcgcgatttaggtacatacaaaacttggtaatcaagaactttcccaacaagttcaactcggaaaacaaagtgattcagtttgaggttaactggaaaaaaatttctacggcaatatacggggatgccaaaaacggcgtgtttaataaagattatgatttcgggtttggaaaagttaggcaggtgaaggacttgcaaatgggactccttatgtatttgggcaaaccaaagcatcatcaccatcaccactga

SEQ ID NO: 4 - protein, 252 aa, Saponaria officinalis
TSITLDLVNPTAGQYSSFVDKIRNNVKDPNLKYGGTDIAVIGPPSKEKFLRINFQSSRGTVSLGLKRDNLYVVAYLAMDNTNVNRAYYFRSEITSAESTALFPEATTANQKALEYTEDYQSIEKNAQITQGDQSRKELGLGIDLLSTSMEAVNKKARVVKDEARFLLIAIQMTAEAARFRYIQNLVIKNFPNKFNSENKVIQFEVNWKKISTAIYGDAKNGVFNKDYDFGFGKVRQVKDLQMGLLMYLGKPK

SEQ ID NO: 5 - protein, 15 aa, Artificial Sequence
  Description of Artificial Sequence Synthetic peptide
GGGGSGGGGSGGGGS

SEQ ID NO: 6 - protein, 298 aa, Artificial Sequence
  Description of Artificial Sequence Synthetic polypeptide
FNKEQQNAFYEILHLPNLNEEQRNAFIQSLKGGGGSGGGGSGGGGSTSITLDLVNPTAGQYSSFVDKIRNNVKDPNLKYGGTDIAVIGPPSKEKFLRINFQSSRGTVSLGLKRDNLYVVAYLAMDNTNVNRAYYFRSEITSAESTALFPEATTANQKALEYTEDYQSIEKNAQITQGDQSRKELGLGIDLLSTSMEAVNKKARVVKDEARFLLIAIQMTAEAARFRYIQNLVIKNFPNKFNSENKVIQFEVNWKKISTAIYGDAKNGVFNKDYDFGFGKVRQVKDLQMGLLMYLGKPK

SEQ ID NO: 7 - protein, 19 aa, Artificial Sequence
  Description of Artificial Sequence Synthetic peptide
MEFGLSWLFLVAILKGVQC

SEQ ID NO: 8 - protein, 6 aa, Artificial Sequence
  Description of Artificial Sequence Synthetic 6xHis tag
HHHHHH

SEQ ID NO: 9 - protein, 15 aa, Artificial Sequence
  Description of Artificial Sequence Synthetic peptide
GSSSSGSSSSGSSSS

(19) United States
(12) Patent Application Publication
(10) Pub. No.: US 2021/0163547 A1
(43) Pub. Date: Jun. 3, 2021

(54) NOVEL SYSTEMS FOR SCREENING INTERNALIZING ANTIBODY AND USES THEREOF

(71) Applicant: Ab Studio Inc., Hayward, CA (US)

(72) Inventors: Yue Liu, Foster City, CA (US); Jiadong Shi, Fremont, CA (US)

(21) Appl. No.: 17/145,870

(22) Filed: Jan. 11, 2021

Related U.S. Application Data
(63) Continuation of application No. PCT/US2019/042314, filed on Jul. 18, 2019.
(60) Provisional application No. 62/700,331, filed on Jul. 19, 2018.

(51) Int. Cl. C07K 14/31; C12N 9/24; G01N 33/533; G01N 33/68

(52) U.S. Cl. CPC C07K 14/31; C12N 9/2497; C12Y 302/02022; G01N 33/533; G01N 2500/10; C07K 2319/02; C07K 2319/21; C07K 2319/55; C07K 2319/705; G01N 33/6854

(57) ABSTRACT

A composition for charactering an internalizing antibody is provided. The composition includes an antibody-binding moiety and a detectable agent.

### RELATED APPLICATIONS

This application is a continuation of International Patent Application PCT/US2019/042314, filed on Jul. 18, 2019, which is related to and claims priority of U.S. Provisional Application No. 62/700,331, filed on Jul. 19, 2018. The entire contents of the foregoing applications are expressly incorporated herein by reference.

### SEQUENCE LISTING

The instant application contains a Sequence Listing which has been submitted electronically in ASCII format and is hereby incorporated by reference in its entirety. Said ASCII copy, created on Jan. 6, 2021, is named 129866-00102_SL.txt and is 11,086 bytes in size.

### FIELD OF THE INVENTION

The present invention provides a composition including an antibody binding moiety and a detectable agent, which can be used in characterizing internalization of an antibody. The compositions may comprise fusion proteins, polypeptides, antibodies, nucleic acids and/or small molecule compositions and/or conjugates thereof.

Further provided are methods, kits and assays for exploiting the provided composition.

### BACKGROUND OF THE INVENTION

For the development of therapeutic antibody drugs, it is often important to identify antibodies that are internalized into cells efficiently, rather than just bind to antigens on the cell surface. Efficient antibody internalization is also important for the delivery of cytotoxic drugs into target cells. Thus, it is important to accurately characterize the efficiency of antibody internalization. Current methods of characterizing antibody internalization either needs to purify the antibody for labeling/conjugation or involve a secondary antibody/Fab conjugated to a detectable agent. The first situation limits the capacity of high throughput screening on un-purified primary antibodies (such as growth media of hybridoma) and the second situation has the following concern: As shown in FIG. 1, upon the binding of the secondary antibody to the primary antibody, the secondary antibody and detectable agent are co-internalized with the primary antibody. The internalization is characterized by detecting and/or measuring the detectable agent or measure cell death caused by internalization of the detectable agent. However, the binding of the secondary antibody/Fab can sometimes induce internalization of the primary antibody that would have not occurred due to the large size of the antigen-primary antibody-secondary antibody/Fab conjugates size (https://en.wikipedia.org/wiki/Endocytosis) and distort the characterization of the internalization efficiency. Thus, there is currently a need for a better system to more accurately detect, measure and/or characterize the internalization efficiency of an antibody.

### BRIEF SUMMARY OF THE INVENTION

The present invention provides compounds, compositions, methods and kits and assays for characterizing antibody internalization.

In one aspect, the present invention provides a composition including an antibody-binding moiety and a detectable agent. In one embodiment, the composition further includes a linker. In another embodiment, the composition includes a fusion protein, wherein the fusion protein includes the antibody-binding moiety and the detectable agent. In another embodiment, the fusion protein includes a linker.

In various embodiments of the above aspects or any other aspect of the invention delineated herein, the antibody-binding moiety is a first peptide or protein and the detectable agent is a second peptide or protein.

In various embodiments of the above aspects or any other aspect of the invention delineated herein, the linker is a third peptide or protein.

In various embodiments of the above aspects or any other aspect of the invention delineated herein, the antibody-binding moiety is a peptide or protein that binds to one or more conserved domains of an antibody. In another embodiment, the conserved domain of the antibody is selected from the group consisting of the Fc domain, the constant domains of the heavy chain (C_{H}1, C_{H}2, C_{H}3), the constant domain of the light chain (C_{L}), the framework regions in the heavy chain variable domain, and the framework regions in the light chain variable domain of the antibody. In yet another embodiment, the conserved domain of the antibody is the Fc domain of the antibody. In still another embodiment, the first peptide or protein is selected from the group consisting of Protein A, Protein G, Protein L, Protein Z, Protein LG, Protein LA, Protein AG, Fc receptor 1 (FcR1), FcR2a, FcR2b, FcR3, FcR4, FcRn (neonatal Fc receptor), an antibody-binding antibody, or an antibody-binding fragment or variant thereof. In another embodiment, the first peptide has the amino acid sequence

| | |
|---|---|
| FNKEQQNAFYEILHLPNLNEEQRNAFIQSLK. | (SEQ ID NO: 1) |

In various embodiments of the above aspects or any other aspect of the invention delineated herein, the second peptide or protein is selected from the group consisting of cytotoxic proteins and fluorescent protein, and fragments or variants thereof. In one embodiment, the cytotoxic protein is selected from the group consisting of ricin (e.g., deglycosylated ricin A chain (dgA)), abrin, mistletoe lectin, or modeccin) or hemitoxin (class I ribosome-inactivating proteins, e.g., PAP, saporin, bryodin 1, bouganin, or gelonin), or fragments or variants thereof that retain cytotoxic activity. In another embodiment, the fluorescent protein is selected from the group consisting of Sirius, Azurite, EBFP2, TagBFP, mTurquoise, CFP, ECFP, Cerulean, TagCFP, mTFP1, mUkG1, mAG1, AcGFP1, TagGFP2, EGFP, GFP, mWasabi, EmGFP, YFP, TagYPF, EYFP, Topaz, SYFP2, Venus, Citrine, mKO, mKO2, mOrange, mOrange2, RFP, TagRFP, TagRFP-T, mStrawberry, mRuby, mCherry, mRaspberry, mKate2, mPlum, mNeptune, T-Sapphire, mAmetrine, mKeima. In yet another embodiment, the cytotoxic protein is saporin (SEQ ID NO: 4).

In various embodiments of the above aspects or any other aspect of the invention delineated herein, the linker has the amino acid sequence GGGGSGGGGSGGGGS (SEQ ID NO: 5).

In various embodiments of the above aspects or any other aspect of the invention delineated herein, the fusion protein includes a signal peptide. In one embodiment, the signal peptide is a prokaryotic signal peptide. In another embodiment, the signal peptide has the amino acid sequence MEFGLSWLFLVAILKGVQC (SEQ ID NO: 7).

In various embodiments of the above aspects or any other aspect of the invention delineated herein, the fusion protein comprises a protein tag. In one embodiment, the protein tag is selected from the group consisting of a histidine tag, a FLAG tag, a myc tag, an HA tag, a GST tag, Calmodulin tags, FLAG tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like. In another embodiment, the protein tag is the histidine tag having the amino acid sequence HHHHHH (SEQ ID NO: 8). In another embodiment, the fusion protein has the amino acid sequence of SEQ ID NO: 2.

In one aspect, the present invention provides a polyncleotide encoding any protein or peptide of any above aspects. In one embodiment, the polynucleotide encodes the fusion protein having the amino acid sequence of SEQ ID NO: 6. In another embodiment, the polynucleotide has the nucleic acid sequence of SEQ ID NO: 3.

In one aspect, the present invention provides an expression vector comprising a polynucleotide according to various embodiments of the above aspects or any other aspect of the invention delineated herein. In another embodiment, the expression vector comprises a polynucleotide having the nucleic acid sequence of SEQ ID NO: 3.

In one aspect, the present invention provides an host cell comprising a vector according to various embodiments of the above aspects or any other aspect of the invention delineated herein. In one embodiment, the host cell is a prokaryotic cell. In another embodiment, the host cell is an E. coli cell.

In one aspect, the present invention provides a method of charactering an internalizing antibody, comprising: contacting the antibody with a cell; contacting the antibody with a composition comprising an antibody-binding moiety and a detectable agent; and characterizing the detectable agent in the cell. In one embodiment, the composition comprising a fusion protein according to any one of above aspects. In another embodiment, the detectable agent is a cytotoxic protein. In yet another embodiment, the cytotoxic protein is a saporin (SEQ ID NO: 4). In still another embodiment, the detectable agent is characterized by a cytotoxicity assay. In still another embodiment, the cytotoxicity assay is selected from 7-AAD assay and calcein releasing assay. In another embodiment, the antibody is selected from the group consisting of a purified antibody, a hybridoma supernatant, and an antibody containing medium.

In one aspect, the present invention provides a method of screening an internalizing antibody, comprising contacting a cell with a plurality of antibodies; contacting the antibody with a composition comprising an antibody-binding moiety and a detectable agent; and detecting the detectable agent in the cell. In one embodiment, the composition comprising a fusion protein according to various embodiment of any of the preceding aspects. In another embodiment, the detectable agent is a cytotoxic protein. In yet another embodiment, the cytotoxic protein is a saporin (SEQ ID NO: 4). In still another embodiment, the detectable agent is character. rized by a cytotoxicity assay. In yet another embodiment, the cytotoxicity assay is selected from 7-AAD assay and calcein releasing assay. In another embodiment, the plurality of antibodies are selected from a group of purified antibodies, supernatants of a plurality of hybridoma cultures, and antibodies or an antigen binding fragment thereof expressed on the surface of a phage display library.

In one aspect, the present invention provides a kit for detecting, measuring and/or characterizing an internalizing antibody, comprising a composition according to various embodiments of the above aspects or any other aspect of the invention delineated herein.

In one embodiment, the fusion protein has the amino acid sequence of SEQ ID NO: 6.

In one embodiment, the polynucleotide encodes the fusion protein having the amino acid sequence of SEQ ID NO: 2.

Other features and advantages of the invention will be apparent from the following detailed description and the drawings.

### BRIEF DESCRIPTION OF THE DRAWINGS

FIG. 1 is a schematic illustrating a traditional high-throughput method of charactering an internalizing antibody. A primary antibody, such as an antibody produced by hybridoma, binds to a cell surface antigen of a cell, such as a disease cell (for example, a cancer cell). A secondary antibody conjugated with a detectable agent, such as a toxin, binds to the primary antibody. The secondary antibody and the detectable agent is co-internalized with the primary antibody-cell surface antigen complex. The internalizing antibody can be characterized by an assay that detects cell death.

FIG. 2 is a schematic illustrating an exemplary embodiment of the present invention. In this exemplary embodiment, a method of the invention characterizes an internalizing antibody without a secondary antibody. A composition of the invention comprises an antibody-binding moiety, such as a peptide, and a detectable agent, such as a toxin. The composition of the invention binds to a primary antibody that binds to a cell surface antigen. The composition of the invention is co-internalized with the primary antibody-cell surface antigen complex. The internalizing antibody can be characterized by an assay that detects and/or measures the detectable agent or the biological consequence caused by the agent in the cell.

FIG. 3 is an image showing the results of an analysis applying Schrodinger BioLuminate software (Schrödinger, LLC, New York, N.Y.). The figure contains the sequence (SEQ ID: 1) that includes the residues of Protein A that are important for the binding of Protein A to the Fc domain of IgG.

FIG. 4 is an image showing the structure of an exemplary fusion protein of the present invention. The fusion protein includes a signal peptide, an antibody (the sequence shown in FIG. 3), a linker, a cytotoxic protein, and a protein tag.

FIG. 5 depicts the sequence of the fusion protein (SEQ ID NO: 2) in FIG. 4. The signal peptide sequence is in italic font, the antibody sequence is in bold font, the linker sequence is underlined, the cytotoxic protein sequence is both bold and underlined, and the protein tag is in regular font.

FIG. 6 depicts an exemplary polynucleotide sequence (SEQ ID NO: 3) that encodes the fusion protein in FIG. 4. The same font representation as used in FIG. 5 applies to the corresponding coding sequences in FIG. 6.

FIG. 7 is a schematic illustrating how an exemplary fusion protein of the invention is used in a method of characterizing an internalizing antibody. The fusion protein is designated “PEP-ZAP” (see below herein for explanation). PEP-ZAP is a fusion protein comprising a Fc binding peptide (PEP) conjugated to a translation inhibitor protein (ZAP). After binding to an internalizing antibody, PEP-ZAP is co-internalized with the antibody and causes cell death, which is the readout of antibody internalization.

FIG. 8 is a graph depicting the effect of internalized PEP-ZAP on cell. Sk-BR-3 cells, a breast cancer cell line, were used in the study. In both control group and test group of cell cultures, medium alone or medium containing Herceptin, Rituxan, or IgG1 isotype immunoglobulin was added in the culture, respectively. In the control group, medium without PEP-ZAP was added to the culture. In the test group, medium containing PEP-ZAP was added to the culture. The results showed that Herceptin alone caused cell death. PEP-ZAP specifically enhanced the death causing effect of Herceptin due to internalization of PEP-ZAP.

FIG. 9 is a graph depicting PEP-ZAP mediated cell death of SIHA.

FIG. 10 is a graph depicting antibody internalization after 24 hours incubation with SIHA as tested by pHrodo assay.

FIG. 11 is a graph depicting internalization ability of different antibodies to PANC1 cells as tested by PEP-ZAP assay.

### DETAILED DESCRIPTION OF THE INVENTION

The present invention is based, at least partly, on the discovery that a polypeptide comprising an antibody binding moiety conjugated to a detectable agent was efficiently co-internalized with an antibody that binds a cell surface antigen and is internalized into the cell. The invention features compositions, systems and methods of detecting, screening, measuring and/or characterizing internalizing antibodies.

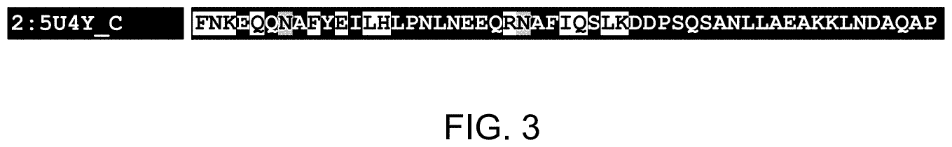

FIG. 2 depicts an exemplary embodiment of the invention. In certain embodiments, a composition of the invention comprises an antibody-binding moiety and a detectable agent. In some embodiments, the antibody-binding moiety is a peptide that binds to an antibody and the detectable agent is a toxin. In certain embodiments, a method of the invention involves contacting a cell with an antibody, and contacting the antibody with the composition of the invention. In some embodiments, the composition contacting the antibody comprises a peptide antibody-binding moiety. In some embodiments, the detectable agent is a toxin. Upon the internalization of the antibody-cell surface antigen complex, the detectable agent is co-internalized into the cell. One skilled in the art can readily characterizing the internalizing antibody by detecting and/or measuring the detectable agent located in the cell using methods well known in the art.

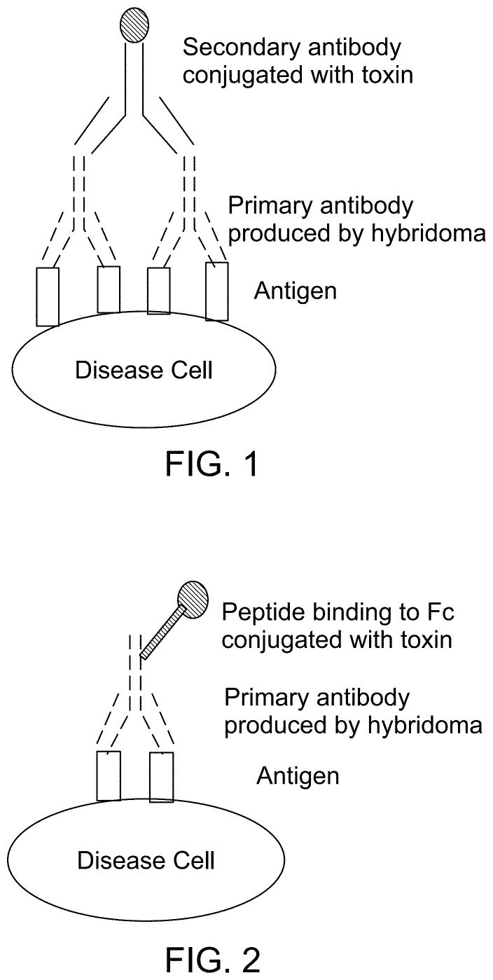

The methods according to the present invention offer several advantages over the traditional methods as shown in FIG. 1. The present invention avoids the use of a secondary antibody. The composition of the invention can be designed to be much smaller compared to a secondary antibody. As a result, the composition of the invention can access a primary antibody-cell surface antigen complex in a manner that more accurately reflects the “real” binding of an antibody to a cell surface antigen. Further, size of the cell surface complex is a factor for antibody internalization. Accordingly, the composition of the invention is less likely to induce an artificial antibody internalization due to the increased size of the complex caused by the binding of the composition to the primary antibody-cell surface antigen complex compared to the traditional methods. Moreover, the preparation of a secondary antibody conjugated with a detectable agent is usually more expensive. The production of an secondary antibody is usually carried out in an eukaryotic cell culture. By contrast, in certain embodiments of the present invention, a composition of the invention, such as a fusion protein, can be produced in a prokaryotic cell culture, such as an E. coli culture.

### I. Antibody Internalization

Upon the binding to its cell surface antigen, some antibody-antigen complexes are internalized into cells. Such an antibody is an internalizing antibody. The internalization depends on, among other things, the characteristics of the cell surface antigen, the antibody, and the size of the complex. Internalization of an antibody is particularly important for antibody-drug conjugate (ADC) based chemotherapy (Dan, N et al., Antibody-Drug Conjugates for Cancer Therapy: Chemistry to Clinical Implications, Pharmaceuticals, 2018, 11, 32). A site-specific targeted delivery of cytotoxic drugs is proving to be a better option for efficient drug delivery. This can be achieved by conjugating cytotoxic drugs to a suitable and validated mAb. ADC strategy not only enhances the therapeutic window of potent cytotoxic drugs, but also minimizes chemo-associated side effects. ADCs attain the idea of a “magic bullet” conceptualized by Paul Ehrlich. The concept of ADC in drug development was well recognized following the Food and Drug Administration (FDA) approval for Adcetris® (brentuximab vedotin) in 2011 and Kadcyla® (trastuzumab emtansine) in 2013. These successes prompted enormous interest among antibody guided therapeutic researchers from both academia and industry. (Dan, supra).

As used herein, the term “antigen” means a substance that has the ability to induce a specific immune response. Examples of antigen include, but are not protein fragments or peptides, DNA, oligonucleotide, polysaccharide, and lipids.

ADCs typically include a fully humanized mAb targeting an antigen specifically/preferentially expressed on tumor cells, a cytotoxic payload, and a suitable linker. Upon binding to the specific antigen, the antibody gets absorbed through rapid internalization followed by lysosomal degradation, and subsequently releasing the cytotoxic drug inside the cell. This way, ADCs can be used to deliver cytotoxic drugs to cancer cells. Selection of mAbs for generation of ADCs is based on their tumor penetrating ability and binding affinity (Kd<10 nM). (Dan, supra)

Internalization of ADC takes place via endocytosis. Endocytosis can be manifested by different internalization routes such as clathrin-mediated, caveolae-mediated, and clathrin-caveolin-independent endocytosis (Kalim et al., Intracellular trafficking of new anticancer therapeutic: antibody-drug conjugates, Drug Design, Development and Therapy, 2017, 11 2265-76). The first two are receptor-mediated endocytosis, while the latter one is receptor-independent endocytosis. High binding interaction of antigen-antibody results in accumulation of more ADCs on the membrane surface. It is hard to distinguish among these pathways as some molecules are not restricted to a single pathway. Trafficking of ADCs occurs with the aid of adaptor protein (AP2), dynamin, epsin, and phosphatidylinositol (4,5) bi-phosphate (PIP2) (Kalim, supra).

Identification and characterization of an internalizing antibody are thus important in the development of cancer drugs, especially ADCs. The present invention provides a platform that can be applied to screen and characterize antibodys with cancer specific internalization/penetration ability.

### II. Compositions of the Invention

The present invention features a composition that comprises an antibody binding moiety and a detectable agent. The detectable agent is attached to the antibody binding moiety. Upon the co-internalization of the antibody and the composition of the present invention binding to the antibody, one of ordinary skill in the art can readily characterize the internalization by detecting and/or measuring the detectable agent located within a cell using any methods disclosed herein or well known in the art.

In certain embodiments, the composition of the present invention further comprises a linker. In some embodiments, the detectable agent is attached to the antibody-binding moiety directly. In some embodiments, the detectable agent is attached to the antibody-binding moiety indirectly. For example, the detectable agent is attached to the antibody-binding moiety through the linker. In some embodiments, the detectable agent is attached to the antibody-binding moiety or linker covalently. In some embodiments, the detectable agent is attached to the antibody-binding moiety or linker non-covalently.

### Antibody-Binding Moiety

The antibody-binding moiety binds to an antibody and does not substantially interfere with the antibody's binding to a target cell surface antigen and internalization. The antibody-binding moiety can be any chemical entity that binds to an antibody. An antibody-binding moiety can include, but is not limited to an atom, a chemical group, a nucleoside, a nucleotide, a nucleobase, a sugar, a nucleic acid, an amino acid, a peptide, a polypeptide, a protein, or a protein complex. In certain embodiments, an antibody-binding moiety binds to an antibody but does not substantially affect one or more functions of the antibody. For example, an antibody-binding moiety may bind to an antibody but does not substantially affect the antibody's ability to bind a cell surface antigen and to be internalized. In certain embodiments, the antibody-binding moiety binds to one or more conserved domains of an antibody. Examples of the conserved domains of the antibody include, but are not limited to, Fc-domain, the constant domains of the heavy chain (C_{H}1, C_{H}2, C_{H}3), the constant domain of the light chain (C_{L}), the framework regions in the heavy chain variable domain, and the framework regions in the light chain variable domain.

In certain embodiments, the antibody binding moiety is a peptide/protein that binds to an antibody. The peptide/protein may bind to one or more conserved domains of an antibody that share sequence similarities between different antibodies and may be capable of binding to a variety of antibodies.

In certain embodiments, the antibody binding peptide/protein is derived from a protein or a peptide that binds to an antibody. Non limiting examples of the antibody binding proteins or peptides include Protein A, Protein G, Protein L, Protein Z, Protein LG, Protein LA, Protein AG, Fc receptor 1 (FcR1), FcR2a, FcR2b, FcR3, FcR4, FcRn (neonatal Fc receptor), antibody-binding antibody (such as a secondary antibody) or an antibody-binding fragment or variant thereof.

In any of the antibody binding peptides/proteins described herein, one or more conservative amino acid substitutions can be introduced where such substitutions are not likely to substantially decrease the binding affinity between the antibody and the peptide/protein. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

As used herein, the term “variant” refers to a biomolecule resembling a protein or a peptide in structure and/or function comprising some differences in their amino acid sequence, composition or structure as compared to the protein or peptide. In certain embodiments, a variant has an amino acid sequence shares at least about 70%, about 80%, about 90% or more similarity with the protein or peptide. In some embodiments, a variant has an amino acid sequence that shares at least about 85%, about 86%, about 87%, about 88%, about 89%, about 90%, about 91%, about 92%, about 93%, about 94%, about 95%, about 96%, about 97%, about 98%, about 99% similarity with the protein or peptide. In certain embodiments, a variant may have increased or decreased ability, property or quality with respect to one or more particular functions of the protein or peptide. For example, a variant of an antibody with known amino acid sequence may have higher internalization efficiency compared to the antibody.

In certain embodiments, the composition of the present invention comprises a peptide (termed “PEP”) having the amino acid sequence: FNKEQQNAFYEILHLPNLNEEQRNAFIQSLK (SEQ ID NO: 1) and its variants. An antibody binding protein/peptide variant refers to a biomolecule resembling an antibody binding protein or peptide in structure and/or function comprising some differences in their amino acid sequence, composition or structure as compared to the antibody binding protein or peptide. A variant may have an increased or decreased binding affinity to the antibody.

In certain embodiments, the PEP variant has an amino acid sequence that shares at least about 70%, about 80%, about 90% or more similarity with SEQ ID NO: 1. In some embodiments, the PEP variant has an amino acid sequence that shares at least about 85%, about 86%, about 87%, about 88%, about 89%, about 90%, about 91%, about 92%, about 93%, about 94%, about 95%, about 96%, about 97%, about 98%, about 99% similarity with SEQ ID NO: 1.

### Detectable Agent

The composition of the invention comprises a detectable agent. The detectable agent co-internalized in a cell can be readily detected or measured by methods well known in the art including, but not limited to, radiography, fluorescence, chemiluminescence, enzymatic activity, absorbance and the like.

In some embodiments, the detectable agent may be a cytotoxin. A cytotoxin or cytotoxic agent includes any agent that may be detrimental to cells. The cytotoxin may be conjugated to the antibody-binding moiety or the linker. Upon the co-internalization of the internalizing antibody and the composition of the present invention comprising a cytotoxin, a cell is killed by the cytotoxin. The internalization efficiency of the antibody can be characterized by detecting and/or measuring the cell death using methods well known in the art. Examples of cytotoxin include, but are not limited to, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020 incorporated herein in its entirety), rachelmycin (CC-1065, see U.S. Pat. Nos. 5,475,092, 5,585,499, and 5,846,545, all of which are incorporated herein by reference), and analogs or homologs thereof.

In some embodiments, the cytotoxin is a cytotoxic protein. In some embodiments, the cytotoxic protein is a plant toxin, e.g., a plant holotoxin (e.g., class II ribosome-inactivating proteins such as ricin (e.g., deglycosylated ricin A chain (dgA)), abrin, mistletoe lectin, or modeccin) or hemitoxin (class I ribosome-inactivating proteins, e.g., PAP, saporin, bryodin 1, bouganin, or gelonin), or fragments or variants thereof that retain cytotoxic activity. See, e.g., Neville et al., J Contr Rel., 1993; 24:133-141; Vallera, Blood, 1994; 83:309-317; Vitetta et al., Immunology Today, 1993; 14:252-259; Kreitman et al., AAPS J., 2006; 8(3):E532-E551).

In some embodiments, the cytotoxic protein is saporin and its variant. Saporin is a ribosome inactivating protein (RIP) due to its N-glycosidase activity. Saporin is isolated from the seeds of Saponaria officinalis. In some embodiments, saporin (designated as “SAP”) has the amino acid sequence:

(SEQ ID NO: 4)
TSITLDLVNPTAGQYSSFVDKIRNNVKDPNLKYGGTDIAVIGPPSKEKFL
RINFQSSRGTVSLGLKRDNLYVVAYLAMDNTNVNRAYYFRSEITSAESTA
LFPEATTANQKALEYTEDYQSIEKNAQITQGDQSRKELGLGIDLLSTSME
AVNKKARVVKDEARFLLIAIQMTAEAARFRYIQNLVIKNFPNKFNSENKV
IQFEVNWKKISTAIYGDAKNGVFNKDYDFGFGKVRQVKDLQMGLLMYLGK
PK.

In certain embodiments, the composition of the present invention comprises a SAP variant. In certain embodiments, the SAP variant has an amino acid sequence that shares at least about 70%, about 80%, about 90% or more similarity with SEQ ID NO: 4. In some embodiments, the SAP variant has an amino acid sequence that shares at least about 85%, about 86%, about 87%, about 88%, about 89%, about 90%, about 91%, about 92%, about 93%, about 94%, about 95%, about 96%, about 97%, about 98%, about 99% similarity with SEQ ID NO: 4.

In certain embodiments, the detectable agent may be one or more of various organic small molecules, inorganic compounds, nanoparticles, enzymes or enzyme substrates, fluorescent materials, luminescent materials (e.g., luminol), bioluminescent materials (e.g., luciferase, luciferin, and aequorin), chemiluminescent materials, radioactive materials (e.g., ¹⁸F, ⁶⁷Ga, ^{81m}Kr, ⁸²Rb, ¹¹¹In, ¹²³I, ¹³³Xe, ²⁰¹Tl, ¹²⁵I, ³⁵S, ¹⁴C, ³H, or ^{99m}Tc (e.g., as pertechnetate (technetate(VII), TcO₄⁻)), and contrast agents (e.g., gold (e.g., gold nanoparticles), gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons).

In certain embodiments, the detectable agent is a fluorescent polypeptide (e.g., GFP or a derivative thereof such as enhanced GFP (EGFP)) or a luciferase (e.g., a firefly, Renilla, or Gaussia luciferase). It will be appreciated that, in certain embodiments, a detectable agent may react with a suitable substrate (e.g., a luciferin) to generate a detectable signal. Non-limiting examples of fluorescent proteins include GFP and derivatives thereof, proteins comprising chromophores that emit light of different colors such as red, yellow, and cyan fluorescent proteins, etc. Exemplary fluorescent proteins include, e.g., Sirius, Azurite, EBFP2, TagBFP, mTurquoise, CFP, ECFP, Cerulean, TagCFP, mTFP1, mUkG1, mAG1, AcGFP1, TagGFP2, EGFP, mWasabi, EmGFP, YFP, TagYPF, EYFP, Topaz, SYFP2, Venus, Citrine, mKO, mKO2, mOrange, mOrange2, RFP, TagRFP, TagRFP-T, mStrawberry, mRuby, mCherry, mRaspberry, mKate2, mPlum, mNeptune, T-Sapphire, mAmetrine, mKeima. See, e.g., Chalfie, M. and Kain, S R (eds.) Green fluorescent protein: properties, applications, and protocols (Methods of Biochemical Analysis, v. 47). Wiley-Interscience, Hoboken, N.J., 2006, and/or Chudakov et al., Physiol Rev. 90(3):1103-63, 2010 for discussion of GFP and numerous other fluorescent or luminescent proteins. In some embodiments, a detectable agent comprises a dark quencher, e.g., a substance that absorbs excitation energy from a fluorophore and dissipates the energy as heat.

In some embodiments, the detectable agent may be a non-detectable precursor that becomes detectable upon activation (e.g., fluorogenic tetrazine-fluorophore constructs (e.g., tetrazine-BODIPY FL, tetrazine-Oregon Green 488, or tetrazine-BODIPY TMR-X) or enzyme activatable fluorogenic agents (e.g., PROSENSE® (VisEn Medical))). In vitro assays in which the enzyme labeled compositions can be used include, but are not limited to, enzyme linked immunosorbent assays (ELISAs), immunoprecipitation assays, immunofluorescence, enzyme immunoassays (EIA), radioimmunoassays (RIA), and Western blot analysis.

### Linker

In certain embodiments, the composition of the invention further comprises a linker. As used herein, a linker refers to a moiety that connects two or more domains, moieties or entities. In one embodiment, a linker may comprise 10 or more atoms. In a further embodiment, a linker may comprise a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. In some embodiments, a linker may comprise one or more nucleic acids comprising one or more nucleotides. In some embodiments, the linker may comprise an amino acid, peptide, polypeptide or protein. In some embodiments, a moiety bound by a linker may include, but is not limited to an atom, a chemical group, a nucleoside, a nucleotide, a nucleobase, a sugar, a nucleic acid, an amino acid, a peptide, a polypeptide, a protein, a protein complex, a payload (e.g., a therapeutic agent) or a marker (including, but not limited to a chemical, fluorescent, radioactive or bioluminescent marker). The linker can be used for any useful purpose, such as to form multimers or conjugates.

In certain embodiments, the composition of the present invention comprises a peptide/protein linker. In certain embodiments, the peptide/protein linker is flexible to maintain the natural folding status of the linked proteins or peptides. In certain embodiments, the peptide/protein linker has the amino acid sequence:

| | |
|---|---|
| GGGGSGGGGSGGGGS. | (SEQ ID NO: 5) |

### Fusion Protein

The invention provides chimeric or fusion proteins comprising an antibody-binding moiety peptide/protein and a detectable agent peptide/protein. As used herein, a “chimeric protein” or “fusion protein” comprises all or part (preferably a biologically active part) of the antibody-binding moiety peptide/protein operably linked to all or part (preferably a biologically active part) of the detectable agent peptide/protein. Within the fusion protein, the term “operably linked” is intended to indicate that the antibody-binding moiety peptide/protein or fragment thereof and the detectable agent peptide/protein or fragment thereof are fused in-frame to each other. The detectable agent peptide/protein or fragment thereof can be fused to the amino-terminus or the carboxyl-terminus of the antibody-binding moiety peptide/protein or fragment thereof.

In certain embodiments, the fusion protein further comprises a linker peptide/protein. The linker peptide/protein may be operably linked at its two termini to both the antibody-binding moiety peptide/protein and the detectable agent peptide/protein. The linker peptide/protein can be fused to the amino-terminus or the carboxyl-terminus of the antibody-binding moiety peptide/protein or fragment thereof. The linker peptide can also be fused to the amino-terminus or the carboxyl-terminus of the detectable agent peptide/protein or fragment thereof.

In certain embodiments, the fusion protein has the amino acid sequence:

(SEQ ID NO: 6)
FNKEQQNAFYEILHLPNLNEEQRNAFIQSLKGGGGSGGGGSGGGGSTSIT
LDLVNPTAGQYSSFVDKIRNNVKDPNLKYGGTDIAVIGPPSKEKFLRINF
QSSRGTVSLGLKRDNLYVVAYLAMDNTNVNRAYYFRSEITSAESTALFPE
ATTANQKALEYTEDYQSIEKNAQITQGDQSRKELGLGIDLLSTSMEAVNK
KARVVKDEARFLLIAIQMTAEAARFRYIQNLVIKNFPNKFNSENKVIQFE
VNWKKISTAIYGDAKNGVFNKDYDFGFGKVRQVKDLQMGLLMYLGKPK.

In some embodiments, the fusion protein contains a heterologous signal peptide having a signal sequence at its amino terminus. For example, the gp67 secretory sequence of the baculovirus envelope protein can be used as a heterologous signal sequence (Ausubel et al., ed., Current Protocols in Molecular Biology, John Wiley & Sons, N Y, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra).

A signal peptide can be used to facilitate secretion and isolation of the fusion proteins. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. The signal sequence directs secretion of the protein, such as from a prokaryotic host into which the expression vector is transformed, and the signal sequence is subsequently or concurrently cleaved. The protein can then be readily purified from the extracellular medium by art recognized methods.

In certain embodiments, the fusion protein comprises a signal peptide having the amino acid sequence MEFGLSWLFLVAILKGVQC (SEQ ID NO:7) and its variants.

In certain embodiments, the fusion protein can also include any type of protein tag that may be useful for purifying the fusion protein. Examples of protein tags include but are not limited to a histidine tag, a FLAG tag, a myc tag, an HA tag, a GST tag, Calmodulin tags, FLAG tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like and their variants.

The protein tag can be fused to the amino terminus of the fusion protein. The protein tag can also be fused to the carboxyl terminus of the fusion protein. In certain embodiments, the protein tag has the amino acid sequence HHHHHH (SEQ ID NO: 8).

In certain embodiments, the fusion protein comprises a signal peptide, an antibody-binding moiety, a detectable agent, and a protein tag. In certain embodiments, the fusion protein has the amino acid sequence:

(SEQ ID NO: 2)
MEFGLSWLFLVAILKGVQCFNKEQQNAFYEILHLPNLNEEQRNAFIQSLK
GGGGSGGGGSGGGGSTSITLDLVNPTAGQYSSFVDKIRNNVKDPNLKYGG
TDIAVIGPPSKEKFLRINFQSSRGTVSLGLKRDNLYVVAYLAMDNTNVNR
AYYFRSEITSAESTALFPEATTANQKALEYTEDYQSIEKNAQITQGDQSR
KELGLGIDLLSTSMEAVNKKARVVKDEARFLLIAIQMTAEAARFRYIQNL
VIKNFPNKFNSENKVIQFEVNWKKISTAIYGDAKNGVFNKDYDFGFGKVR
QVKDLQMGLLMYLGKPKHHHHHH.

The present invention provides a polynucleotide sequence that encodes any of the proteins and/or peptides, or fragments or variants thereof as described herein. In certain embodiments, the polynucleotide sequence encodes an antibody-binding moiety, or fragments or variants thereof. In certain embodiments, the polynucleotide sequence encodes a detectable agent, or fragments or variants thereof. In certain embodiments, the polynucleotides sequence comprises a sequence that encodes the antibody-binding moiety, or fragments or variants thereof and a sequence that encodes the detectable agent, or fragments or variants thereof. In certain embodiments, the polynucleotide sequence comprises a sequence that encodes a signal peptide, or fragments or variants thereof. In certain embodiments, the polynucleotide sequence comprises a sequence that encodes a protein tag, or fragments or variants thereof. In some embodiments, the polynucleotide sequence comprises a sequence that encodes the fusion protein having the amino acid sequence of SEQ ID NO: 6. In some embodiments, the polynucleotide sequence comprises a sequence that encodes the fusion protein having the amino acid sequence of SEQ ID NO: 2.

In certain embodiments, the polynucleotide sequence comprises:

(SEQ ID NO: 3)
atggagtttgggctgagctggctttttcttgtggctattttaaaaggtgt
ccagtgtttcaacaaagaacaacaaaacgccttctatgaaatcttgcact
tgcctaacttgaacgaagaacaacgcaatgctttcatccaaagcttaaaa
ggtggaggcggttcaggtggaggcggttcaggtggaggcggttcaACATC
AATCACATTAGATCTAGTAAATCCGACCGCGGGTCAATACTCATCTTTTG
TGGATAAAATCCGAAACAACGTAAAGGATCCAAACCTGAAATACGGTGGT
ACCGACATAGCCGTGATAGGCCCACCTTCTAAAGAAAAATTCCTTAGAAT
TAATTTCCAAAGTTCCCGAGGAACGGTCTCACTTGGCCTAAAACGCGATA
ACTTGTATGTGGTCGCGTATCTTGCAATGGATAACACGAATGTTAATCGG
GCATATTACTTCAGATCAGAAATTACTTCCGCCGAGTCAACCGCCCTTTT
CCCAGAGGCCACAACTGCAAATCAGAAAGCTTTAGAATACACAGAAGATT
ATCAGTCGATTGAAAAGAATGCCCAGATAACACAAGGAGATCAAAGTAGA
AAAGAACTCGGGTTGGGGATTGACTTACTTTCAACGTCCATGGAAGCAGT
GAACAAGAAGGCACGTGTGGTTAAAGACGAAGCTAGATTCCTTCTTATCG
CTATTCAGATGACGGCTGAGGCAGCGCGATTTAGGTACATACAAAACTTG
GTAATCAAGAACTTTCCCAACAAGTTCAACTCGGAAAACAAAGTGATTCA
GTTTGAGGTTAACTGGAAAAAAATTTCTACGGCAATATACGGGGATGCCA
AAAACGGCGTGTTTAATAAAGATTATGATTTCGGGTTTGGAAAAGTTAGG
CAGGTGAAGGACTTGCAAATGGGACTCCTTATGTATTTGGGCAAACCAAA
Gcatcatcaccatcaccactga

The present invention provides a recombinant vector that expresses any of the proteins and/or peptides, or fragments or variants thereof as described herein. In certain embodiments, the recombinant expression vector comprises any one of the polynucleotides as described herein. Non-limiting examples of such expression vectors are the pUC series of vectors (Fermentas Life Sciences), the pBluescript series of vectors (Stratagene, La Jolla, Calif.), the pET series of vectors (Novagen, Madison, Wis.), the pGEX series of vectors (Pharmacia Biotech, Uppsala, Sweden), and the pEX series vectors (Clontech, Palo Alto, Calif.).

Chimeric and fusion proteins of the invention can be produced by standard recombinant DNA techniques. In certain embodiments, the fusion gene can be synthesized by conventional techniques including automated DNA synthesizers. In certain embodiments, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra).

The general methodology for making any of the proteins or peptides as described herein is well known in the art. Standard techniques may be used for recombinant DNA, oligonucleotide synthesis, transformation and protein production. Enzymatic reactions, protein productions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. supra), which is incorporated herein by reference for any purpose.

“Transformation”, as defined herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The present invention provides a recombinant host cell that expresses the proteins or peptides, or the fragments and variants thereof as described herein. The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Examples of host cells include, but are not limited to, prokaryotic and eukaryotic cells selected from any of the Kingdoms of life. Examples of eukaryotic cells include, but are not limited to, protist, fungal, plant and animal cells. Non-limiting examples of host cells include, but are not limited to, the prokaryotic cell E. coli; mammalian cell lines CHO, HEK 293 and COS; the insect cell line Sf9; and the fungal cell Saccharomyces cerevisiae.

### III. Methods and Uses

### Characterization of Antibody Internalization

Compositions and methods of the present invention may be used to characterize the internalization induced by a wide variety of antibody-cell surface antigen binding. The present invention provides composition that comprise an antibody-binding moiety that can binds to a variety antibodies and a detectable agent. In certain embodiments, to characterize the internalization induced by antibody-cell surface antigen binding, an antibody to be tested contacts a cell. An suitable amount of the composition of the invention contacts the antibody. The internalization of the antibody-cell surface antigen complex is characterized by detecting and/or measuring the detectable agent that is co-internalized into the cell. In certain embodiments, the composition comprises a fusion protein that comprises a peptide/protein antibody-binding moiety and a peptide/protein detectable agent.

It is well within the ability of one skilled in the art to adjust various parameters of the methods of the invention to characterize the internalization efficiency of an antibody. For example, in connection with the antibody binding to the cell surface antigen and internalization, one skilled in the art can readily adjust the amount of the antibody, the incubation time, the incubation temperature, and/or other parameters to increase the antibody binding and to decrease the internalization of the antibody-cell surface antigen complex before the binding of the composition of the invention to the antibody. One skilled in the art can adjust any one of such parameters, or any combination of parameters, to characterize the internalization of the antibody.

In certain embodiments, the antibody to be characterized is a purified antibody. In certain embodiments, the antibody to be characterized is an antibody in an antibody containing medium or hybridoma growth supernatant. In certain embodiments, the antibody to be characterized is an antibody or an antigen-binding fragment thereof on the surface of a phage in a phage display library. The antibody in an antibody containing medium or hydridoma growth supernatant can be partially purified and/or concentrated using well known methods in the art. For example, the medium containing the antibody or hydridoma growth supernatant can be filtered to increase the concentration of the antibody, or dialyzed to remove one or more small molecules.

To detect, measure and/or characterize the antibody internalization, any probes may be used in concert with any of the compositions, kits, or methods disclosed herein. As used herein, the term “probe” refers to any molecule that may bind or associate, indirectly or directly, covalently or non-covalently, to any substrates and/or products caused by the detectable agent disclosed herein and whose association or binding is detectable using the methods disclosed herein or methods well known in the art. In some embodiments, the probe is a fluorogenic, fluorescent, or chemiluminescent probe, an antibody, or an absorbance-based probe. A probe may be immobilized, adsorbed, or otherwise non-covalently bound to a solid surface.

In some embodiments, the detectable agent causes a biological change in the nature or chemical availability of one or more targets the probes may bind or associate. For instance, if the internalized detectable agent caused cell death, DNA in the cell may become available to a fluorescent probe that binds to the DNA. The internalization can thus be detected, measured and characterized using fluorescence based assay for the fluorescent probe. The intensity, length, or amplitude of a wavelength emitted from the fluorescent agent can be measured and is, in some embodiments, proportional to the presence, absence or quantity of the detectable agent internalized into the cell. The quantity of the internalized detectable agent can be determined from detection of the intensity of or fluorescence at a known wavelength of light using methods well known in the art. In certain embodiments, FACS (Fluorescence-activated cell sorting) may be used in a fluorescence based assay to characterize the antibody internalization by detecting and/or measuring the detectable agent.

In certain embodiments, the internalized detectable agent is detected and/or measured without a probe. For example, the detectable agent may be a fluorescent agent. The quantity of the internalized detectable agent can be determined from detection of the intensity of fluorescence at a known wavelength of light directly.

It is well within the ability of one skilled in the art to detect, measure and/or characterize the detectable agent that is co-internalized with the antibody-cell surface antigen complex. The detectable agent can be detected, measured and/or characterized using any methodologies well known in the art. For example, if the internalized detectable agent causes cell death, one skilled in the art can readily choose one of many available cell death assays to detect, measure and/or characterize the cell death.

In certain embodiments, the invention provides a methods to detect, measure and/or characterize the antibody internalization using a cytotoxicity assay such as cell death assay. In certain embodiments, the cell death is characterized using 7-amino actinomycin-D (7-AAD) assay. In certain embodiments, the cell death is characterized using calcein releasing assay.

The efficiency of the internalization of an antibody is detected, measured, and/or characterized compared to a “reference” or “control.” As used herein, a “reference” or “control” refers to a baseline measured in a comparable assay. For example, a “reference” or “control” may be an otherwise identical assay for an antibody except that the antibody used in the “reference” or “control” assay is known to be unable to induce internalization.

The efficiency of the internalization of an antibody may be compared to a “reference” or “control antibody.” As used herein, a “reference” or “control antibody” refers to an antibody that has a known internalization efficiency. An antibody has a higher internalization efficiency if the antibody has a statistically significant improvement in its internalization efficiency compared to a reference or control antibody.

### Screening of Internalizing Antibodies

The present invention provides methods to implement strategies for developing internalizing antibody. In certain embodiments, the present invention provides a method of screening and characterization of internalizing antibodies for a known cell surface target antigen. A suitable subject, such as a mouse, can be immunized with an antigen. The antigen can be the extracellular domain of a receptor, an antigen overexpressing cell, and/or target antigen gene. The antibody titer in the immunized subject can be monitored over time by standard techniques, such as with an enzyme linked immunosorbent assay (ELISA). At an appropriate time after immunization, e.g., when the specific antibody titers are highest, antibody-producing cells can be obtained from the subject and used to prepare monoclonal antibodies (mAb) by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody are screened by using the composition of the invention to screen the hybridoma culture supernatants for antibodies that bind the target cell surface antigen.

In certain embodiments, the present invention provides a method of screening internalizing antibodies using the fusion protein of the invention. Cells, such as disease cells (for example, cancer cells), having the target cell surface antigen are incubated together with a plurality of antibodies and the composition of the invention. In some embodiments, the plurality of antibodies are purified antibody. In certain embodiments, the plurality of antibodies are antibodies in an antibody containing medium. In certain embodiments, the plurality of antibodies are antibodies in the supernatants of a plurality of hybridoma cultures. In certain embodiments, the plurality of antibodies are antibody or an antigen binding fragment thereof expressed on the surface of a phage in a phage display library. In certain embodiments, the composition of the invention comprises the fusion protein of the invention comprising an antibody-binding moiety and a detectable agent. In some embodiments, the fusion protein binds to a conserved domain of the antibody. In certain embodiments, the fusion protein binds to the Fc domain of the antibody. The internalization of an antibody into the cell, such as a disease cell (for example, a cancer cell), can be detected by detecting the effect of the detectable agent in the cell. In certain embodiments, the detectable agent is a cytotoxic protein. The internalization of the antibody can be detected based on cytotoxicity. In certain embodiments, the fusion protein of the invention comprises the PEP antibody-binding moiety and the cytotoxic protein saporin (SAP). The screening methods can include one or more reference or control antibody. The internalizing antibody having internalization efficiency significantly higher than the reference or control antibody is subject to further characterization using any of the assay methods of the invention or well known in the art.

In certain embodiments, the assay to screen internalizing antibody involves the following steps:

1) Plate target cells on a 96 well plate
2) Add purified antibody or transfection medium containing antibody or hybridoma growth supernatant containing antibody to target cells
3) After incubation for an appropriate period of time (e.g., 1 hour), 4) Add cell culture media containing an appropriate amount of PEP-ZAP in an appropriate concentration (e.g., 10 μg/mL) into the 96 well plate and incubate for an appropriate amount of time (e.g., 4 hours or overnight)
5) Check cell death rate by staining with an appropriate method (e.g., stained with 7AAD and analyzed by FACS; or alternatively, target cells being stained with calcein, and cell death being analyzed by calcein release).

More cell death represents better antibody internalization based, at least partly, on the following mechanism of action: After target cells are bound by testing antibodies, addition of PEP-ZAP will lead to binding of PEP-ZAP to the Fc domain of testing antibodies, because PEP is the Fc binding domain on protein A. If testing antibody is internalized, Fc bound PEP-ZAP will be co-internalized and serve as a translation inhibitor inside the target cell and lead to cell death.

In certain embodiments, the present invention provides a method of identifying a cell surface target antigen if the target antigen is unknown. To identify a target antigen, cells, such as disease cells (for example, cancer cells), are incubated with a phage library for human naïve single chain variable fragment (ScFV) or Fab. The incubation is carried out under a condition that does not induce internalization, such as at 4° C. After removing any non-“binders,” the cells are incubated under a condition (for example, at 37° C.) to induce internalization. The “binders” that bind to the cell surface antigen but are not internalized are removed using a special buffer (50 mlvi glycine, 150 mm NaCl, 200 mlvi urea, and 2 mg/ml polyvinylpyrrolidone, pH 2.8, http://cancerres.aacrjournals.org/content/64/2/704.long: As used here, the term “binder” refers to a phage that express a ScFv or Fab on its surface that binds to a cell surface antigen. The cells are lysed and fresh E. coli cells are infected by the phage containing cell lysates are used to produce more “binder” phages that produce internalizing ScFv or Fab. The resulting phage mixture is further subject to more rounds (for example, three (3) rounds) of panning. The leading “binders” are selected and are subject to sequencing. The ScFv or Fab gene in the “binder” phage is converted to an IgG immunoglobulin gene for further characterization. A co-immunoprecipitation is carried out to capture IgG version of the leading “binder” and the antigen it recognizes. Mass-spectrum is carried out to identify the antigen.

In certain embodiments, foregoing assay methods of the invention are amenable to high-throughput screening (HTS) implementations. In some embodiments, the screening assays of the invention are high throughput or ultra high throughput (see, e.g., Fernandes, P. B., Curr Opin Chem. Biol. 1998, 2:597; Sundberg, S A, Curr Opin Biotechnol. 2000, 11:47). High throughput screens often involve testing large numbers of antibodies with high efficiency, e.g., in parallel. For example, tens or hundreds of thousands of antibodies can be routinely screened in short periods of time, e.g., hours to days. In some embodiments, HTS refers to testing of between 1,000 and 10,000 antibodies per screening. In some embodiments, ultrahigh throughput refers to screening in excess of 10,000 antibodies per screening, e.g., up to 1,000,000 or more antibodies per screening. The screening assays of the invention may be carried out in a multi-well format, for example, a 96-well, 384-well format, 1,536-well format, or 3,456-well format and are suitable for automation. In some embodiments, each well of a microwell plate can be used to run a separate assay against a different test antibody, or, if concentration or incubation time effects are to be observed, a plurality of wells can contain test samples of a single antibody, with at least some wells optionally being left empty or used as controls or replicates. Typically, HTS implementations of the assays disclosed herein involve the use of automation. In some embodiments, an integrated robot system including one or more robots transports assay microwell plates between multiple assay stations for compound, cell and/or reagent addition, mixing, incubation, and readout or detection. In some aspects, an HTS system of the invention may prepare, incubate, and analyze many plates simultaneously. Suitable data processing and control software may be employed. High throughput screening implementations are well known in the art. Without limiting the invention in any way, certain general principles and techniques that may be applied in embodiments of a HTS of the present invention are described in Macarron R & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Exemplary methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006).

### IV. Kits

Any of the compositions described herein may be comprised in a kit. In a non-limiting example, reagents for detecting, measuring, and characterizing antibody internalization, including compositions of the invention are included in a kit. In certain embodiments, the fusion protein comprising the antibody-binding moiety and the detectable agent is included. The kit may further include reagents or instructions for antibody internalization assays. It may also include one or more buffers. Other kits of the invention may include components for the assays to detect the detectable agent. In certain embodiments, the kits of the invention comprise the reagents for assaying cell death. In certain embodiments, the kits of the invention comprise the reagents for 7-ADD cell death assay. In certain embodiments, the kits of the invention comprise the reagents for calcein release cell death assays.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. The kits may also comprise a second container means for containing a sterile, pharmaceutically acceptable buffer and/or other diluent. However, various combinations of components may be comprised in a vial. The kits of the present invention also will typically include a means for containing the compositions of the invention, e.g., the fusion proteins, and any other reagent containers in close confinement for commercial sale.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

### V. Definitions

In order that the present invention may be more readily understood, certain terms are first defined. In addition, it should be noted that whenever a value or range of values of a parameter are recited, it is intended that values and ranges intermediate to the recited values are also intended to be part of this invention. The following is a non-limiting list of term definitions.

As used herein, the term “about” or “approximately,” as applied to one or more values of interest, refers to a value that is similar to a stated reference value. In certain embodiments, the term “about” or “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “antibody,” as used herein, is intended to refer to immunoglobulin molecules comprised of four polypeptide chains, two heavy (H) chains and two light (L) chains inter-connected by disulfide bonds. Other naturally occurring antibodies of altered structure, such as, for example, camelid antibodies, are also included in this definition. Each heavy chain is comprised of a heavy chain variable region (abbreviated herein as HCVR or VH) and a heavy chain constant region. The heavy chain constant region is comprised of three domains, C_{H}1, C_{H}2 and C_{H}3. Each light chain is comprised of a light chain variable region (abbreviated herein as LCVR or VL) and a light chain constant region. The light chain constant region is comprised of one domain, C_{L}. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. One constant and one variable domain from each heavy and light chain of the antibody form the Fab region of an antibody. Two or three constant domains, depending on the class of the antibody, of the two heavy chains contribute to form the Fc region or Fc domain of the antibody.

As used herein, the term “antibody-binding moiety” refers to any chemical entity that binds to an antibody. An antibody-binding moiety can include, but is not limited to an atom, a chemical group, a nucleoside, a nucleotide, a nucleobase, a sugar, a nucleic acid, an amino acid, a peptide, a polypeptide, a protein, or a protein complex. In certain embodiments, an antibody-binding moiety binds to an antibody but does not substantially affect one or more functions of the antibody. For example, an antibody-binding moiety may bind to an antibody but does not substantially affect the antibody's ability to bind a cell surface antigen and to be internalized.

As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association”, a “link”, or a “conjugation” need not be strictly through direct covalent chemical bonding. It may also suggest ionic or hydrogen bonding or a hybridization based connectivity sufficiently stable such that the “associated” entities remain physically associated.

As used herein, the term “characterize,” “characterizing”, “characterization” and words related thereto refer to the identification and/or quantification of any ability, quality, property or trait of a target, and do not require that the precise chemical identity, e.g., the molecular formula, chemical structure, nucleotide sequence or amino acid sequence, of the target is elucidated. Characterization of the target primarily depends how the target is detected and/or measured. The target can be detected by testing for a property or activity of the target, such as biological property, a chemical property, a physical property, a biochemical property, or a property that is a combination of any of the foregoing. Any biological or chemical property can be assayed that results in a detectable biological or chemical reaction, such as the enzymatic modification of a substrate or cell death. Many of such methods and materials are known in the art.

The term “conserved domain” refers to a set of amino acids conserved at specific positions along an aligned sequence of evolutionally related proteins. While amino acids at other positions can vary between homologous proteins, amino acids that are highly conserved at specific positions indicate amino acids that are important in the structure, the stability, or the activity of a protein. Because they are identified by their high degree of conservation in aligned sequences of a family of protein homologues, they can be used as a site for an agent to recognize multiple members of the identified protein family. For example, of relevance herein, the Fc domain of antibody is a conserved domain in antibodies. A variety of antibodies can be recognized and bound to by certain proteins, such as Protein A.

As used herein, the terms “cytotoxic agent,” or “cytotoxin” refers to an agent that kills or causes injurious, toxic, or deadly effects on a cell (e.g., a mammalian cell (e.g., a human cell)), bacterium, virus, fungus, protozoan, parasite, prion, or a combination thereof.

As used herein, “detectable agent” refers to one or more markers, signals, or moieties which are attached, incorporated or associated with another entity, which markers, signals or moieties are readily detected by methods known in the art including radiography, fluorescence, chemiluminescence, enzymatic activity, absorbance and the like. Detectable agent include radioisotopes, fluorophores, chromophores, enzymes, dyes, metal ions, ligands such as biotin, avidin, streptavidin and haptens, quantum dots, and the like. Detectable agents may be located at any position in the entity with which they are attached, incorporated or associated. For example, when attached, incorporated in or associated with a peptide or protein, they may be within the amino acids, the peptides, or proteins, or located at the N- or C-termini.

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

A “fragment,” as used herein, refers to a portion. For example, fragments of proteins may comprise polypeptides obtained by digesting full-length protein isolated from cultured cells. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids. In some embodiments, fragments of an antibody include portions of an antibody subjected to enzymatic digestion or synthesized as such.

As used herein, the term “isolated” is synonymous with “separated”, but carries with it the inference separation was carried out by the hand of man. In one embodiment, an isolated substance or entity is one that has been separated from at least some of the components with which it was previously associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art. In some embodiments, isolation of a substance or entity includes disruption of chemical associations and/or bonds. In some embodiments, isolation includes only the separation from components with which the isolated substance or entity was previously combined and does not include such disruption.

As used herein, “peptide” is less than or equal to 50 amino acids long, e.g., about 5, 10, 15, 20, 25, 30, 35, 40, 45, or 50 amino acids long.

As used herein, “purify,” means to make substantially pure or clear from unwanted components, material defilement, admixture or imperfection. “Purified” refers to the state of being pure. “Purification” refers to the process of making pure.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1: Identification of Antibody Binding Peptide

A peptide that binds to the Fc domain of IgG was identified by analyzing the binding between Protein A (the Staphylococcus aureus virulence factor protein A) and the Fc domain of IgG. Pdb 5U4Y is a structure formed by the binding of the Fc domain of IgG to the B domain of Protein A. The B domain refers to the immunoglobulin-binding domain found in Protein A. The B domain of Protein A contains three α-helices which are retained upon interaction with the Fc fragment of IgG.

Pdb 5U4Y is a dimer whose structure contains chains A and B, which are the Fc domain of IgG, and chains C and D, which are the B domain of Protein A. The structure of Pdb 5U4Y is available at https://www.rcsb.org/structure/5U4Y. In order to analyze what is the shortest peptide in the B domain of Protein A which interacts with IgG Fc, chains B and D were deleted using Schrodinger BioLuminate software (Schrödinger, LLC, New York, N.Y.) The interaction between chain A (the Fc domain of IgG) and chain C (the B-domain of Protein A) was analyzed using Schrodinger BioLuminate software.

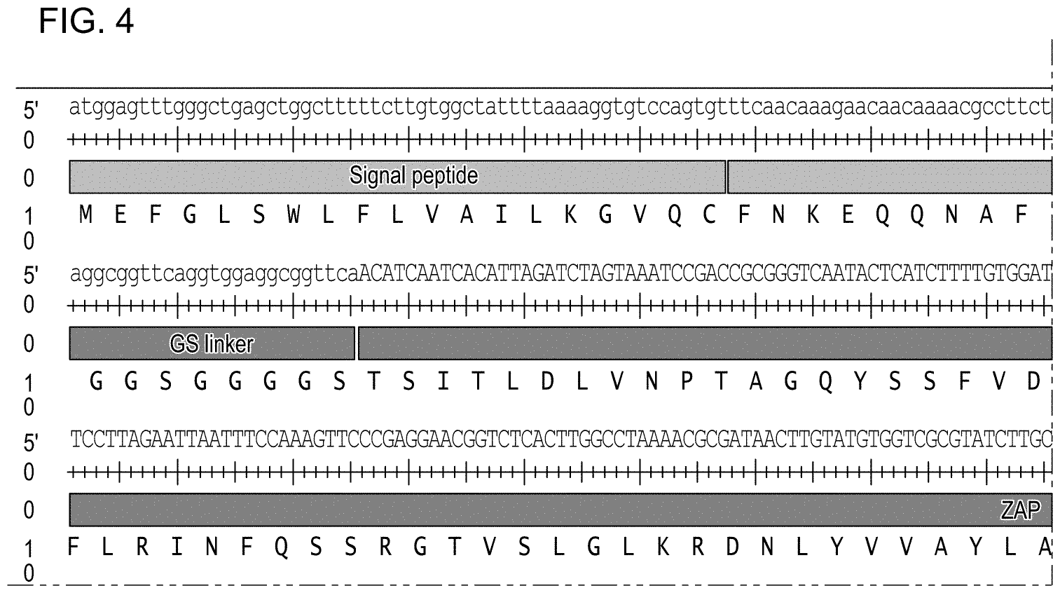

FIG. 3 provides an enlarged view of the analysis result of the Schrodinger BioLuminate software. The peptide with the amino acid sequence FNKEQQNAFYEILHLPNLNEEQRNAFIQSLK (SEQ ID NO: 1) contains all important binding residues (shown in white background in FIG. 3) of chain C. SEQ ID NO: 1 is the shortest peptide identified by now that interacts with the Fc domain of IgG based on the inventors' knowledge.

To assess if this truncated peptide (SEQ ID NO: 1) would lose the natural helix folding as in the B domain of Protein A, the folding of this peptide is analyzed using Schrodinger BioLuminate software. The analysis result shows that this truncated peptide (SEQ ID NO: 1) would not lose the natural helix folding. This peptide is designated as “PEP.”

### Example 2: Design and Structure of Fusion Protein

A PEP-ZAP fusion protein was designed and can be applied for screening antibody with internalization potential. The PEP-ZAP can also be applied for detecting, measuring and characterizing antibody internalization. This fusion protein comprises the following three parts: 1) a peptide which is a region of Protein A interacting with IgG Fc domain (PEP). PEP was selected by analyzing pdb 5U4Y C structure as described in Example 1; 2) “GGGGS”×3 linker (SEQ ID NO: 5); and 3) Saporin (ZAP), a so-called ribosome inactivating protein (RIP), due to its N-glycosidase activity, from the seeds of Saponaria officinalis.

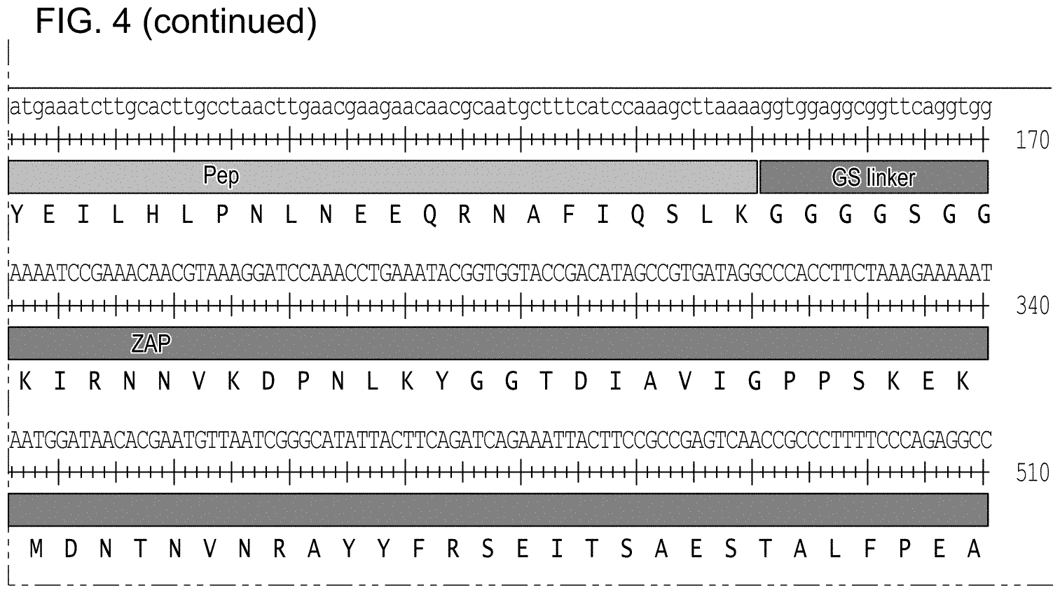

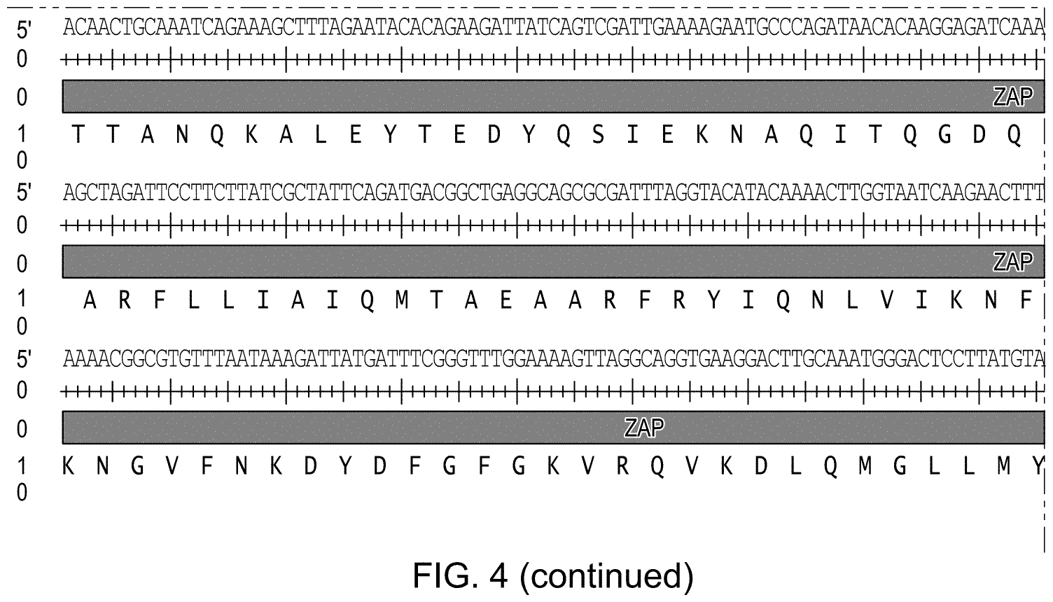

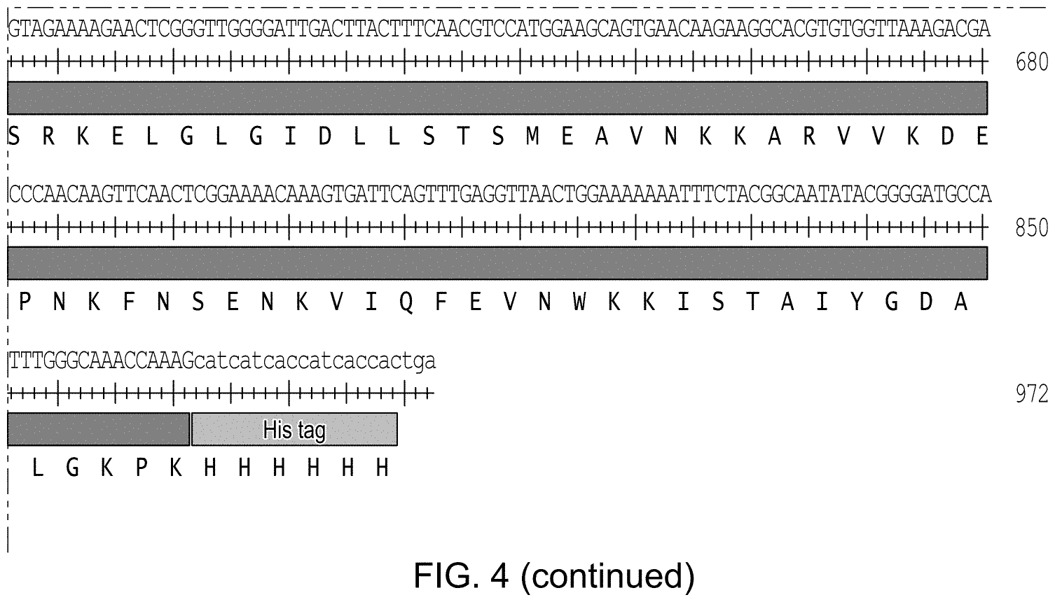

After confirming that PEP retains its natural helix folding, it was applied as the “PEP” part of “PEP-ZAP”. A “GGGGS”×3 linker, which is widely used in single chain fragment of variable region (ScFV), was applied to link PEP and ZAP together. This linker is very flexible to maintain the linked proteins' natural folding status. FIG. 4 illustrates the structure of a fusion protein that comprises PEP-ZAP. The fusion protein as shown in FIG. 4 further comprises a signal peptide at its amino-terminus and a hexa-histidine (6×His) (SEQ ID NO: 8) tag at its carboxyl terminus. In FIG. 4, the signal peptide sequence, the PEP sequence, the GS linker (GGGGS×3 linker (SEQ ID NO: 5)) sequence, the ZAP sequence, and the 6×His tag (SEQ ID NO: 8) are indicated by the bar underneath. FIG. 5 shows the amino acid sequence (SEQ ID NO: 2) of the fusion protein in FIG. 4. The domains are represented by different font. FIG. 6 shows one exemplary polynucleotide sequence (SEQ ID NO: 3) that encodes the fusion protein in FIG. 4. The same font code as used in FIG. 5 applies to the corresponding coding sequences in FIG. 6.

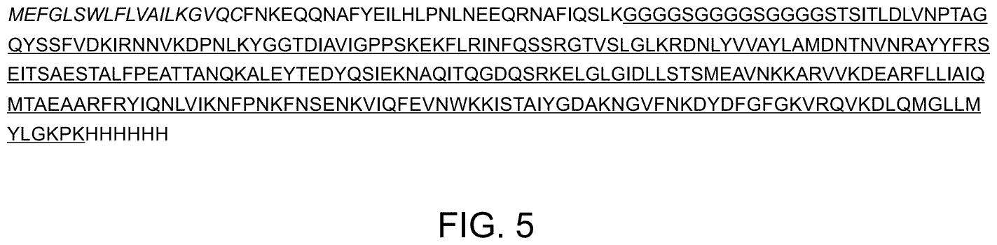

FIG. 7 illustrates how PEP-ZAP works. As described herein, PEP-ZAP is a fusion protein that comprises an Fc binding peptide (PEP) conjugated to a translation inhibitor protein (SAP). PEP-ZAP binds to a primary antibody produced by hybridoma that binds to an antigen present on the surface of a cell, such as a diseased cell (for example, a cancer cell). Upon co-internalization of PEP-ZAP with the cell surface antigen-primary antibody complex, PEP-ZAP causes cell death, which is the readout of antibody internalization.

### Example 3: Internalization of Herceptin in her 2 Positive SK-Br-3 Cancer Cell

SK-Br-3 is a cell line isolated from a breast cancer patient. The cell-line over-expresses the Her 2 gene product. Herceptin (trastuzumab) is a monoclonal antibody that targets the Her 2 gene product, inducing the internalization and downregulation of Her 2 gene. The Herceptin—Her 2 gene product interaction is used herein as an antibody internalization model to evaluate the effect of PEP-ZAP. Rituxan (rituximab), a monoclonal antibody that does not recognize Her 2 gene product, is used herein as a control antibody. An IgG1 immunoglobulin is also used herein as a control. Table 1 shows the experimental design for one replicate in a 96 well plate. One assay includes eight (8) replicates in one 96 well plate.

TABLE 1

| | | | | | |
|---|---|---|---|---|---|
| cells | cells | cells | cells | cells | cells |
| Medium | Medium | Herceptin | Rituxan | isotype | Medium |
| Medium | Medium | Medium | Medium | Medium | Medium |
| cells | cells | cells | cells | cells | cells |
| Medium | Medium | Herceptin | Rituxan | isotype | Medium |
| PEP-ZAP | PEP-ZAP | PEP-ZAP | PEP-ZAP | PEP-ZAP | PEP-ZAP |

For each replicate, PEP-ZAP was present in the medium of a test group (the six wells at bottom row) and absent in the medium of a control group (the six wells at top row). For the test group and the control group, three wells contained medium only, and Herceptin, Rituxan or IgX isotype was present in one well in each group. SK-Br-3 cells were cultured according to the manufacturer's protocol. Cells were detached by trypsinization and suspended in RPMI 1640 with 10% FBS media. The cell concentration was adjusted to about 1×10⁶/ml. Herceptin, Rituxan and IgX isotype were prepared at 20 μg/mL. Fifty micro-liter (50 μL) of cells were dispensed into each well of a flat bottom 96-well plate. Medium containing one hundred micro-liter (100 μL) of antibodies or medium without antibody was added in each well according to the design as shown in Table 1. The cells were incubated at 37° C. for thirty (30) minutes. Fifty micro-liter (50 μL) of medium or medium containing 40 μg/mL PEP-ZAP was added in wells according to the design as shown in Table 1. The cells were incubated at 37° C. for twenty (20) hours. The cells were detached by trypsinization and washed with FACS buffer two times. The cells were then stained with 7-AAD by incubating with 7-ADD at room temperature for twenty (20) minutes. The cells were washed twice with FACS buffer. FACS analysis was run to quantify the cell death.

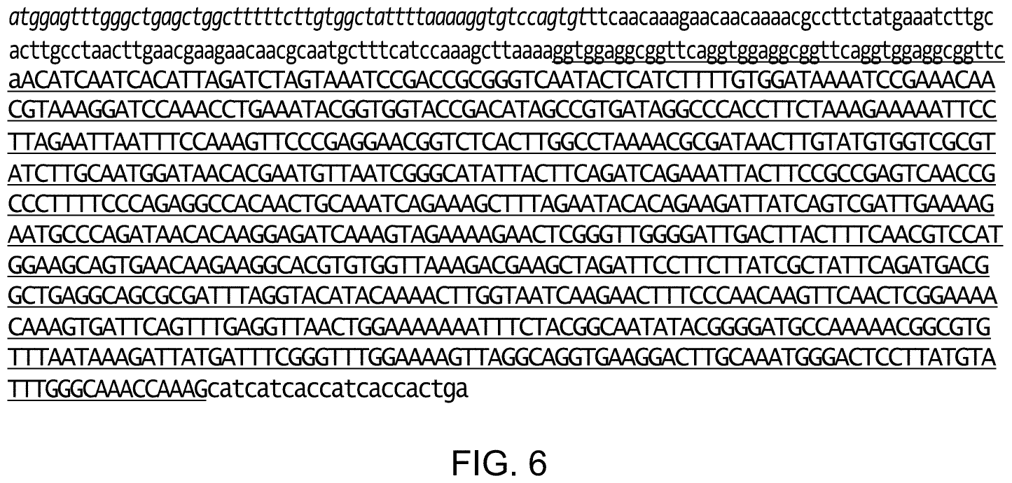

The results of the assay, as shown in FIG. 8, demonstrated that PEP-ZAP specifically binds to an IgG antibody and is specifically co-internalized with an internalizing antibody. In the two controls in the control group (Rituxan, and IgX isotype), addition of the antibodies without PEP-ZAP did not cause increased cell death. Herceptin by itself induced cell death (apoptosis) of Her 2 positive cells. In the test group, the presence of PEP-ZAP did not change the cell death in the two controls (Rituxan and IgX isotype), indicating that PEP-ZAP was not internalized into the cells. On the other hand, when PEP-ZAP was present in Herceptin containing medium, internalization of Herceptin brought PEP-ZAP into Sk-Br-3 cells and led to more cell death as indicated by percentage of 7AAD positive cells.

### Example 4: PD-L1/CD55 Bispecific Antibody Internalization

Programmed death-ligand 1 (PD-L1) plays a major role in suppressing the immune system. A variety of tumor cell lines expressing high levels of PD-L1 and PD-L1 expression is closely related to the degree of malignancy and tumor recurrence rate. Anti-PD-L1 monoclonal antibody can effectively bind to cell surface PD-L1 and activated T cells that results in killing of tumor cells.

CD55, also known as complement decay-accelerating factor, indirectly blocks the formation of the membrane attack complex and inhibits CDC. Antibody against CD55 leads to formation of membrane attack complex and activate membrane cascade which cause cell death.

A PD-L1/CD55 bispecific antibody was developed based on their respective action on immune system. This bispecific antibody can block two tumor immune escape modes at the same time and also activitates the tumor cell killing functions of both a T cell and complement system. The tumor inhibition effect of the bispecific antibody can be significantly improved compared to that of two monospecific antibodies. The PD-L1/CD55 bispecific antibody was described in detail in International Patent Publication No. WO/2019/028125, the contents of which are incorporated herein by reference.

In order to get a better efficacy/safety balance, a couple of versions of PD-L1/CD55 bispecific antibodies were designed. In order to study the internalization ability of these antibodies, PEP-ZAP assay was performed followed by confirmation with pHrodo Red assay.

Mechanism of pHrodo Red Assay

Intracellular pH is one of a cell's essential control switches for regulating critical cell functions. Not only does pH affect enzyme activity and other protein functions, but proton gradients are a critical source of energy for driving cell metabolism. Eukaryotic cells contain a variety of defined compartments with different degrees of acidity: intracellular pH is generally between ˜6.8 and 7.2 in the cytosol and between ˜4.5 and 6.5 in acidic vesicles and organelles. The pH-sensitive pHrodo® dyes are unique pH sensors that undergo a dramatic increase in fluorescence in response to an environmental shift from high to low pH. With a pKa of approximately 6.8, both the pHrodo® Red and pHrodo® Green dyes are useful pH indicators between pH 4 and pH 8, making them ideal for monitoring physiologically relevant pH changes.

Though both PEP-ZAP assay and pHrodo methods could be applied for detecting antibody induced internalization, PEP-ZAP assay does not need to apply purified antibody, therefore, has advantage for pre-screening or high throughput screening of internalizing antibodies.

Antibody Internalization Ability Detected by PEP-ZAP Assay was Confirmed by pHrodo Red Assay

Antibody Internalization Detected by PEP-ZAP Assay

To detect antibody internalization using PEP-ZAP, a protocol based on cell killing by internalized PEP-ZAP was used. Briefly, target cells (SIHA cells or MDA231 cells) were prepared and seeded in 96 well plate at 5×10⁴ cells per well. Antibodies were added at a final concentration of 10 μg/ml. The cells and antibodies were incubated at 37° C. for thirty (30) minutes. PEP-ZAP was added to a final concentration of 10 μg/ml. The plate was incubated at 37° C. for 18 hours or 40 hours. After the incubation, the cells were detached by trypsinization and washed with FACS buffer two times. The cells were then stained with 7-AAD (1:50 dilution) by incubating with 7-ADD at room temperature for fifteen (15) minutes. The cells were washed twice with FACS buffer. FACS analysis was run to quantify the cell death.

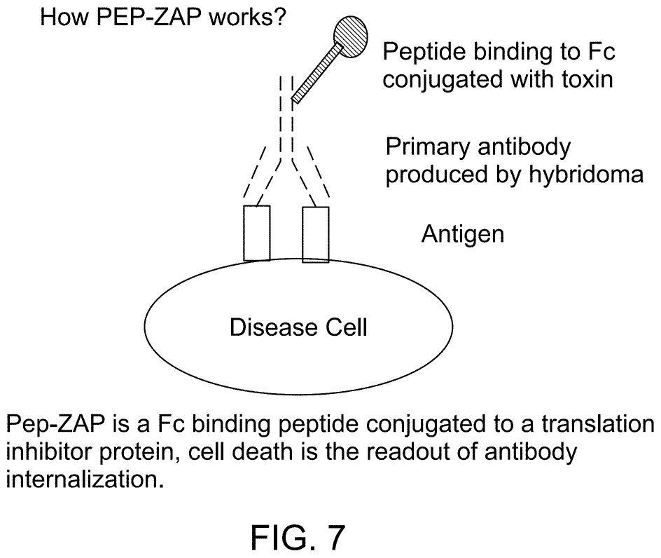

The results of the assay, as shown in FIG. 9, demonstrated that PD-L1/CD55 bispecific antibody induced more cell death than PD-L1 and CD55 antibody in the presence of Pep-ZAP, mainly in SIHA cells but not in MDA231 cells (data not shown). Incubation with the bispecific antibody for longer time (40 hours) caused more cell death than short time (18 hours).

Antibody Internalization Detected by pHrodo Assay

To confirm the internalization detected by PEP-ZAP assay, a protocol based on pHrodo Red intracellular pH indicator was used to detect the internalization of the antibody. Briefly, goat anti-human IgG was labelled with pHrodo Red according to the manufacture's protocol. Target cells (SIHA cells or MDA231 cells) were prepared and seeded in 96 well plate at 5×10⁴ per well. Antibodies were added into the well to the final concentration of 10 μg/ml. The plate was incubated at 37° C. for 30 minutes. pHrodo Red labeled goat against human IgG was added to a final concentration of 10 μg/ml. The plate was incubated at 37° C. for 24 hours. After the incubation, the cells were detached by trypsinization and washed with FACS buffer two times. FACS analysis was run to quantify the signal.

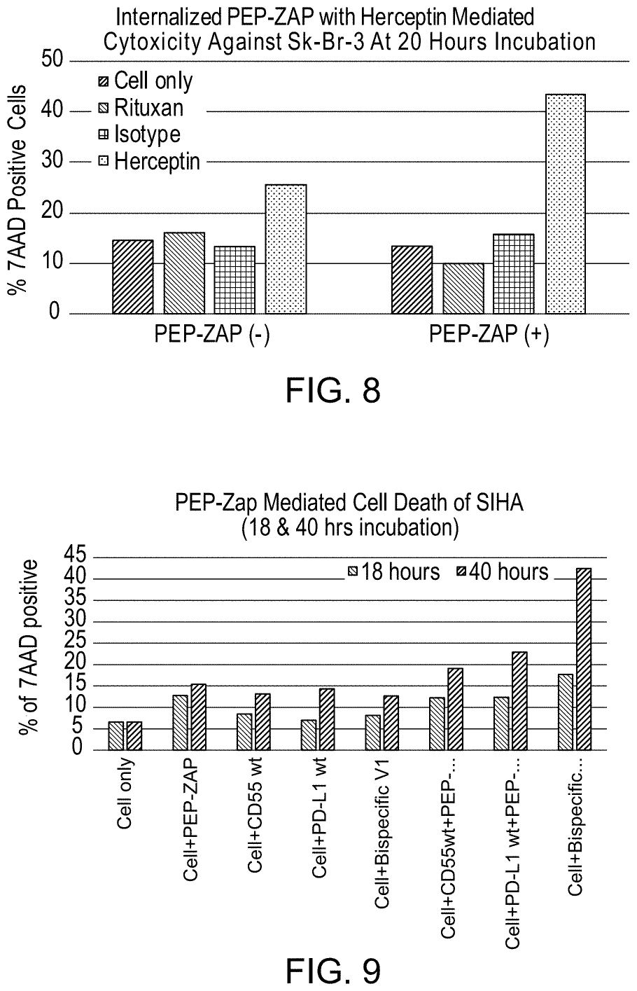

The results of the assay, as shown in FIG. 10, demonstrated that internalization ability of parental CD55 Mab is higher in SIHA cells than PDL1 as judged with pHrodo Red. Further, similar internalization ability of bispecific anti-PDL1xCD55 V1 and V2 was observed. The assay further confirmed that PEP-ZAP assay, described above, reliably detects the internalization of the bispecific antibody just as pHrodo Red.

Screening of New PDL1/CD55 Bispecific Antibodies with Internalizing Ability on Pancreatic Cancer Cell Via PEP-ZAP Assay

As described elsewhere herein, the PEP-ZAP assay is suitable for screening antibodies with internalizing ability. A protocol based on cell killing by internalized PEP-ZAP was used to screen antibodies for internalizing ability. Briefly, target cells (pancreatic cancer cell, PANC1) were prepared and seeded in 96 well plate at 5×10⁴ cells per well. Antibodies were added at a final concentration of 10 μg/ml. The cells and antibodies were incubated at 37° C. for thirty (30) minutes. PEP-ZAP was added to a final concentration of 10 μg/ml. The plate was incubated at 37° C. for 18 hours or 40 hours. After the incubation, the cells were detached by trypsinization and washed with FACS buffer two times. The cells were washed twice with FACS buffer. FACS analysis was run to quantify the signal.

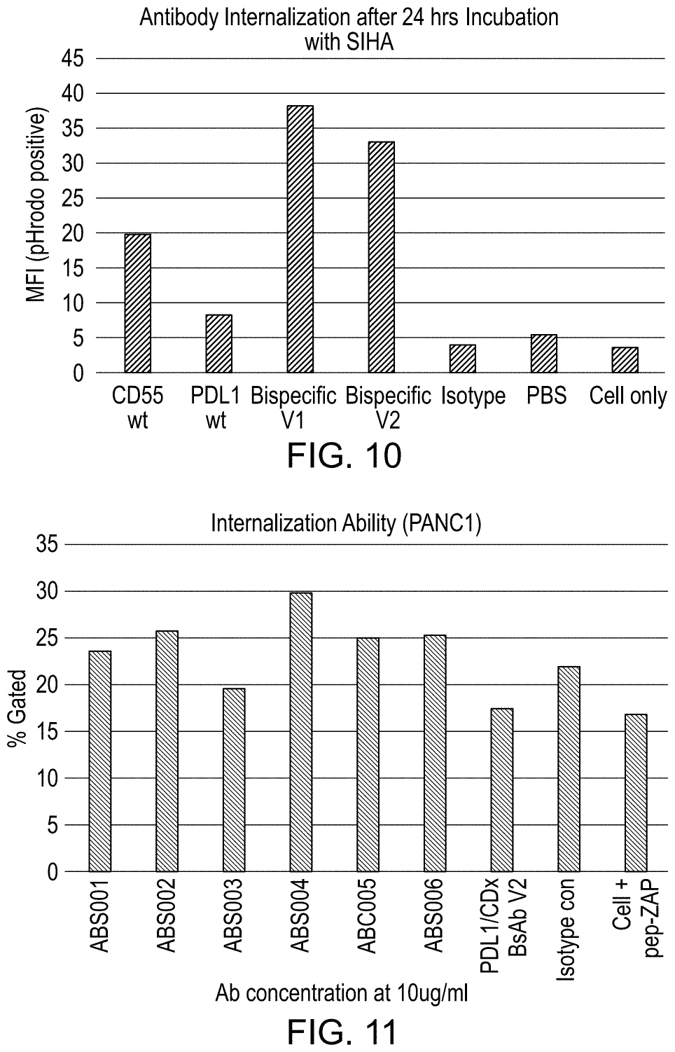

The results, as shown in FIG. 11, demonstrated that multiple novel PDL1/CD55 bispecific antibodies with better internalization ability were observed. The test confirmed that the PEP-ZAP assay can be utilized to screen antibodies with different internalization ability.

### Example 5: Materials and Methods

The following materials and methods were used in Examples 3 and 4.

### Antibodies

Trastuzumab, Rituximab and IgG1 control were prepared in PBS at 20 μg/mL.

### Cell Culture

SK-Br-3 cells were acquired from ATCC. The maintenance and culture of SK-Br-3 cells were carried out according to manufacturer's instruction. Briefly, SK-Br-3 cells were cryopreserved in freezing buffer at −70° C. or in liquid nitrogen. After thawing, the cells were maintained in RPMI 1640 with 10% FBS at 37° C. The cells were passaged when the confluence reached 90%.

### Cell Death Assay

Cell death was assessed using 7-AAD viability assay. 7-ADD was acquired from Thermofisher and the assay was carried out according to the manufacturer's protocol. Briefly, 2 ul of 7-ADD was added in cells in FACS buffer containing PBS with 2.5% FBS. Cells were incubated with 7-ADD for twenty (20) minutes at room temperature. Cells were washed twice with FACS buffer to end the staining.

Cell death was quantified using FACS analysis, which was carried out according to manufacturer's protocol. Briefly, 7AAD fluorescence was detected in FL3 channel, 7AAD positive cells were considered as dead cells.

1. A composition comprising a fusion protein, wherein the fusion protein comprises an antibody-binding moiety and a detectable agent.

2. The composition of claim 1, further comprising a linker.

3. (canceled)

4. (canceled)

5. The composition according to claim 1, wherein the antibody-binding moiety is a first peptide or protein and the detectable agent is a second peptide or protein.

6. The composition according to claim 2, wherein the linker is a third peptide or protein.

7. The composition according to claim 5, wherein the antibody-binding moiety is a peptide or protein that binds to one or more conserved domains of an antibody.

8. The composition according to claim 7, wherein the conserved domain of the antibody is selected from the group consisting of the Fc domain, the constant domains of the heavy chain (CH1, CH2, CH3), the constant domain of the light chain (CL), the framework regions in the heavy chain variable domain, and the framework regions in the light chain variable domain of the antibody.

9. The composition according to claim 7, wherein the conserved domain of the antibody is the Fc domain of the antibody.

10. The composition according to claim 7, wherein the first peptide or protein is selected from the group consisting of Protein A, Protein G, Protein L, Protein Z, Protein LG, Protein LA, Protein AG, Fc receptor 1 (FcR1), FcR2a, FcR2b, FcR3, FcR4, FcRn (neonatal Fc receptor), an antibody-binding antibody, or an antibody-binding fragment or variant thereof.

11. The composition according to claim 10, wherein the first peptide has the amino acid sequence FNKEQQNAFYEILHLPNLNEEQRNAFIQSLK (SEQ ID NO: 1).

12. The composition according to any one of claim 5, wherein the second peptide or protein is selected from the group consisting of cytotoxic proteins and fluorescent protein, and fragments or variants thereof.

13. The composition according claim 12, wherein the cytotoxic protein is selected from the group consisting of ricin (e.g., deglycosylated ricin A chain (dgA)), abrin, mistletoe lectin, or modeccin) or hemitoxin (class I ribosome-inactivating proteins, e.g., PAP, saporin, bryodin 1, bouganin, or gelonin), or fragments or variants thereof that retain cytotoxic activity.

14. The composition according claim 12, wherein the fluorescent protein is selected from the group consisting of Sirius, Azurite, EBFP2, TagBFP, mTurquoise, CFP, ECFP, Cerulean, TagCFP, mTFP1, mUkG1, mAG1, AcGFP1, TagGFP2, EGFP, GFP, mWasabi, EmGFP, YFP, TagYPF, EYFP, Topaz, SYFP2, Venus, Citrine, mKO, mKO2, mOrange, mOrange2, RFP, TagRFP, TagRFP-T, mStrawberry, mRuby, mCherry, mRaspberry, mKate2, mPlum, mNeptune, T-Sapphire, mAmetrine, mKeima.

15. The composition according to claim 13, wherein the cytotoxic protein is saporin (SEQ ID NO: 4).

16. The composition according to claim 2, wherein the linker has the amino acid sequence GGGGSGGGGSGGGGS (SEQ ID NO: 5).

17. The composition according to claim 1, further comprising a signal peptide.

18. The composition according to claim 17, wherein the signal peptide is a prokaryotic signal peptide.

19. The composition according to claim 18, wherein the signal peptide has the amino acid sequence MEFGLSWLFLVAILKGVQC (SEQ ID NO: 7).

20. The composition according claim 1, further comprising a protein tag.

21. The composition according to claim 20, wherein the protein tag is selected from the group consisting of a histidine tag, a FLAG tag, a myc tag, an HA tag, a GST tag, Calmodulin tags, FLAG tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

22. The composition according to claim 21, wherein the protein tag is the histidine tag having the amino acid sequence HHHHHH (SEQ ID NO: 8).

23. The composition according to claim 21, wherein the fusion protein has the amino acid sequence of SEQ ID NO: 2.

24. A polynucleotide encoding the fusion protein of claim 1.

25. (canceled)

26. (canceled)

27. An expression vector comprising a polynucleotide according to claim 24.

28. (canceled)

29. (canceled)

30. A host cell comprising a vector according to claim 27.

31. (canceled)

32. (canceled)

33. A method of charactering an internalizing antibody, comprising:
contacting the antibody with a cell;
contacting the antibody with a composition comprising a fusion protein, wherein the fusion protein comprises an antibody-binding moiety and a detectable agent; and
characterizing the detectable agent in the cell.

34-39. (canceled)

40. A method of screening an internalizing antibody, comprising contacting a cell with a plurality of antibodies;
contacting the antibody with a composition comprising a fusion protein, wherein the fusion protein comprises an antibody-binding moiety and a detectable agent; and
detecting the detectable agent in the cell.

41-46. (canceled)

47. A kit for detecting, measuring and/or characterizing an internalizing antibody, comprising a composition according to claim 1.

48. The composition according to claim 16, wherein the fusion protein has the amino acid sequence of SEQ ID NO: 6.

49. The polynucleotide according the claim 24, wherein the polynucleotide encodes the fusion protein having the amino acid sequence of SEQ ID NO: 2.
